SEQUENCE LISTING (1 sequence)

SEQ ID NO: 1 - DNA, 39 bp, T7 RNA Polymerase
ggccagtgaattgtaatacgactcactatagggaggcgg

(19) United States
(12) Patent Application Publication
(10) Pub. No.: US 2005/0069909 A1
(43) Pub. Date: Mar. 31, 2005

(54) Method to identify and analyze genes having modified expression in activated cells with secretory lysosomes

(75) Inventors: Alisa Kabcenell, Weston, CT (US); Jun Li, Danbury, CT (US); Daniel Rajotte, Danbury, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 10/737,465

(22) Filed: Dec. 16, 2003

Related U.S. Application Data
(60) Provisional application No. 60/437,239, filed on Dec. 31, 2002.

(51) Int. Cl. C12Q 1/68

(52) U.S. Cl. 435/6

(57) ABSTRACT

A method of identifying genes involved in the regulated secretion of cells having secretory lysosomes comprising the steps of: a) exposing experimental cells to an activating agent; b) preparing RNA from said experimental cells at one or more activation phases; c) measuring the level of gene expression in the cells; d) comparing the levels of gene expression of said experimental cells to the level of gene expression in control cells that have not been exposed to an activating agent; e) identifying genes that are up regulated or down regulated in said experimental cells relative to said control cells.

### BACKGROUND OF THE INVENTION

The invention relates to the field of cell biology, and inflammatory diseases and in particular to methods for identification and analysis of genes having modified expression in activated cells with secretory lysosomes.

### BACKGROUND INFORMATION

Cells that contain secretory lysosomes are thought to play a central role in inflammatory and allergic reactions. Numerous cell types are known to have secretory lysosomes including mast cells, goblet cells, neutrophils, natural killer cells, basophils, eosinophils, melanocytes, dendritic cells, macrophages, B cells, osteoclasts and platelets. (Griffiths, (2002) Semin. Cell Dev. Biol. 13: 279-284). All of these cell types undergo activation and release of their secretory lysosomes upon presentation of an appropriate activating agent. As a result of becoming activated these cells are known to release potent inflammatory mediators such as histamine, proteases, chemotactic factors, cytokines and metabolites of arachidonic acid that act on various parts of the body including the vasculature, smooth muscle, connective tissue, mucous glands and inflammatory cells.

Mast cells are a secretory lysosomal cell type that is of particular interest with regard to inflammatory diseases such as asthma. (Williams, et al, (2000) J. Allergy Clin. Immunol., 105: 847-859). Mast cells are found resident in tissues throughout the body, particularly in association with structures such as blood vessels, nerves, and in proximity to surfaces in contact with the external environment (see Metcalfe et al, (1997) Physiol Rev., 77: 1033-1079). Mast cell activation may be initiated upon interaction of a multivalent antigen with its specific IgE antibody attached to the cell membrane via its high affinity receptor, FcεRI. The identification of novel genes or new functions for known genes involved in mast cell related processes such as degranulation, cytokine production, regranulation, and neuro-immune communication is considered important in developing therapeutics approaches to the treatment of diseases such as allergic, inflammatory, immunological and cardiovascular diseases.

The pathways for granule biogenesis and exocytosis in mast cells are still largely obscure. Griffiths, G. M. et al, (1996) Biochem. Biopys. Res. Commun., 222 (3): 802-808, 1996; Masuda et al, (2002) FEBS Lett. 470: 61-64, 2000; Baram, D. et al, (2001) J. Immunol., 167 (7): 4008-4016. Mast cells contain structures known as secretory lysosomes which are a mixture of lysosomes and secretory granules. Stinchcombe and Griffiths, J. Cell Biol. 146 (1): 1-6. The mast cell granule can be described as a modified lysosome, specialized for fusion with the plasma membrane, and with other lysosomal granules, after receptor activation. Although similar secretory lysosomes are found in hematopoietic cells, little is known about the mechanisms by which these organelles receive and deliver their cargo. For example, Riesbeck et al. (WO 98/42850) disclose protein targeting to endothelial cell Weibel-Palade bodies. Weibel-Palade bodies contain the adhesion molecule P-selectin.

There are two categories of inflammatory mediators in mast cells and basophils: preformed mediators and newly formed mediators. Preformed mediators, stored in the cytoplasmic granules of rodent or human mast cells, include histamine, proteoglycans, cytokines, serine proteases, carboxypeptidase A and small amounts of sulfatases and exoglycosidases. Metcalfe et al., (1997) Physiol Rev. 77 (4): 1033-1079, 1997. Histamine acts on a set of receptors (H1, H2, H3, H4) on cells and tissues and is rapidly metabolized extracellularly. Proteoglycans may function to package histamine and basic proteins into secretory granules, and in human mast cells may stabilize the protease tryptase. Neutral proteases, which account for the vast majority of the granule protein, serve as markers of mast cells. Newly formed mediators, often absent in resting mast cells, consist of arachidonic acid metabolites, principally leukotriene C and prostaglandin D. These mediators are typically produced during IgE receptor activation. Of particular interest in humans is the production of tumor necrosis factor (TNF), Interleukin (IL)-4, IL-5 and IL-6.

Proteases are the major protein constituent exocytosed from activated mast cells. Huang et al, (1998) J. Clin. Immunol., 18: 169-183. Tryptases, chymases, and carboxypeptidases are the three major families of proteases stored in the secretory granules and secretory lysosomes of mast cells. Chymases are part of the serine protease family. Immunohistochemical localization indicates that they are only synthesized in mast cells. Beil et al, (2002) Histol Histopathol, 15 (3): 937-946. Human, primate, and dog chymase generate angiotensin II (Ang II) from Ang I, while mouse and rat chymases degrade Ang II. Fukami et al, (1998) Curr Pharm Des. 4 (6): 439-453. Chymase also degrades extracellular matrix, and processes procollagenase, inflammatory cytokines and other bioactive peptides. As a result, chymase plays important roles in inflamed tissues through its proteolytic activities.

In human cells, genes encoding two chymotryptic enzymes (chymase and Cathepsin G-like protease) and one mast cell carboxypeptidase enzyme and at least two genes encoding tryptase peptides have been detected. The gene encoding chymase is closely linked to the gene encoding cathepsin G, an enzyme apparently expressed in mast cells, and to the genes encoding granzymes. Mucosal (MC)-type mast cells contain tryptase, chymase, cathepsin G-like protease and mast-cell carboxypeptidase. The biological function of mast cell neutral proteases, like mast cells themselves, remains to be fully clarified. For example, on-going mast cell activation in asthma appears to be a characteristic of this chronic inflammatory disease.

In murine mast cells, five chymases (Mouse Mast Cell Protease (MMCP)-1, -2, -3, -4, and -5), one mast cell carboxypeptidase and two tryptases (MMCP-6 and -7) have been reported. In rodents, the protease composition of mast cell subsets differs. In rats two isoforms of chymase, Rat Mast Cell Protease (RMCP) I (Lagunoff and Pritzl, (1976) Arch Biochem Biophys., 173 (2): 554-563) and RMCP II (Kido et al, Arch Biochem Biophys. 239 (2): 436-443, 1985) were found to distinguish the mast cells in mucosal surfaces (RMCP-II positive), from other mast cells (RMCP-I positive). Gibson and Miller, (1986) Immunology, 58 (1): 101-104. More recently, two additional serine proteases were isolated by PCR amplification from rat serosal MC: the rat tryptase (the counterpart of MMCP-6) and an additional chymase named RMCP III. Lutzelschwab et al, (1997) J Exp Med. 185 (1): 13-29. The latter protease is the rat counterpart of mouse MMCP-5.

Scientists have reported on a protein Rab37, that can localize to the surface of mast cell granules when fused to green fluorescent protein (GFP) and is expressed in bone-marrow derived mast cells. Masuda et al., FEBS Lett. 470: 61-64. Rab37 appears to localize to the cytoplasmic surface of granules. However, Masuda does not teach the use of Rab37 to target granules.

Genes that have modified expression in cells having secretory lysosomes have been identified by biochemical methods including immunoprecipitation and protein overexpression. SAGE analysis has been used to characterize gene expression in resting and activated mast cells. Chen et al, Journal of Experimental Medicine, 188:1657-1668. In the Chen study, genes such as cytokine macrophage migration inhibitory factor, neurohormone receptors and melatonin were found to be constitutively expressed. Several genes were found to be differentially expressed in response to antigen induced clustering of the FcεRI, including preprorelaxin, mitogen activated protein kinase 3 and the protein phosphatase rVH6.

Microarray is a technique used to analyze the expression of a large number of genes simultaneously. Debouck et al, (July 2002) Genetics 21: 48-50; Current Protocols in Molecular Biology, John Wiley and Sons, July, 2002. Microarray analysis can be performed in a number of different ways. Microarray analysis can be performed with DNA microarrays which contain microscopic spots of about 1 kb DNA sequences representing thousands of genes bound to the surface of glass microscopic slides. Microarray analysis can also be performed with oligonucleotide arrays (DNA chips) or high density nucleotide probes which contain synthetic oligonucleotides representing thousands of gene sequences synthesized on the surface of small areas of a glass slide.

Microarrays can be used to study the expression profiles of cells and tissues of significance in the study of a variety of diseases. Debouck et al, Annu. Rev. Pharmacol. Toxicol. 40: 193-208. Microarray techniques have been used to study the expression profile of mast cells/basophils and eosinophils. Nakajima et al, (2001) Blood 98: 1127-1134. Nakajima focused on identifying genes that are differentially expressed in mast cells versus eosinophils. This approach however did not address questions such as which genes expressed in mast cells contribute to activation in response to IgE/antigen. High density oligonucleotide probe arrays were used to examine the expression of genes selectively transcribed in mast cells. Debouck et al, Nature Genetics 21: 48-50. Saito et al used microarray analysis in a study finding genes that are selectively expressed in activated mast cells to include L-histidine decarboxylase responsible for histamine synthesis, 1-5-hydroxy-prostoglandin dehydrogenase, carboxypeptidase, chymase 1,4-alpha-glucan branching enzyme and clusterin. Saito et al, (2001) International Archives of Allergy Immunology, 125: 1-8. This strategy did not attempt to study the genes/proteins involved in the early or late phase of allergic response and the dynamics of gene expression following mast cell activation.

### BRIEF SUMMARY OF THE INVENTION

The present invention provides a method of identifying and analyzing genes having modified expression in activated cells with secretory lysosomes comprising the steps of:
a) exposing experimental cells to an activating agent;
b) preparing RNA from said experimental cells at one or more activation phases;
c) measuring the level of gene expression in the cells;
d) comparing the levels of gene expression of said experimental cells to the level of gene expression in control cells that have not been exposed to an activating agent;
e) identifying genes that are up regulated or down regulated in said experimental cells relative to said control cells.

Preferred embodiments of the invention include use of the method with cells selected from the list of cell types consisting of mast cells, goblet cells, neutrophils, natural killer cells, basophils, eosinophiles, melanocytes, dendritic cells, macrophages, B cells, osteoclasts and platelets.

In another preferred embodiment, RNA samples are measured from the early, middle and late stages of activation in cells with secretory lysosomes.

The method of the invention can be used for identifying and analyzing genes that may be targets for the development of inhibitor compounds useful in the treatment of immunological, inflammatory and cardiovascular diseases. Such diseases may be treated through the administration of a pharmaceutically acceptable amount of an inhibitor compounds of genes identified and analyzed using the methodology explained herein.

In another embodiment of the invention gene expression is measured using microarray analysis.

### BRIEF DESCRIPTION OF THE DRAWINGS

FIG. 1 shows the release of hexosaminidase in mast cells following administration with an activating agent using the method of the invention.

FIG. 2 shows gene expression profiles of selected genes at 2 h, 6 h and 24 h after administration of an activating agent using the method of the invention.

FIG. 3 shows gene expression profiles of selected genes with peak expression at 2 h after administration of an activating agent using the method of the invention.

FIG. 4 shows gene expression profiles of selected genes at 6 h after administration of an activating agent using the method of the invention.

FIG. 5 shows gene expression profiles of selected genes at 24 h after administration of an activating agent using the method of the invention.

### DETAILED DESCRIPTION OF THE INVENTION

Unless defined otherwise, the scientific and technological terms and nomenclature used herein have the same meaning as commonly understood by a person of ordinary skill in the art to which this invention pertains. The procedures for cell culture and general molecular biology methods and the like are common methods used in the art. Current Protocols in Molecular Biology, John Wiley and Sons, July, 2002.
Tissue—refers to one or more cells, extracts and fractions thereof.
Cell—refers to cells in any form, including but not limited to, cells retained in tissue, cell clusters and individually isolated cells.
Mast Cell—refers to cells of immunological origin that can be found resident in tissues throughout the body, particularly in association with structures such as blood vessels, nerves, and in proximity to surfaces in contact with the external environment. Mast cells express the FcεERI I receptor and typically release potent inflammatory mediators such as histamine, proteases, chemotactic factors and metabolites of arachidonic acid that act on the vasculature, smooth muscle, connective tissue, mucous glands and inflammatory cells.
Gene transcription refers to a process whereby one strand of a DNA molecule is used as a template for synthesis of a complementary RNA by RNA polymerase.
Gene expression refers to the process whereby information encoded in a particular gene is decoded into a particular protein. The level of gene expression as the term is used herein can be can be determined by measuring the level of mRNA in a cell.
DNA refers to polynucleotide molecules, segments or sequences and is used herein to refer to a chain of nucleotides, each containing the sugar deoxyribose and one of the four adenine (A), guanine (G) thymine (T) or cytosine (C).
RNA refers to polynucleotide molecules, segments or sequences and is used herein to refer to a chain of nucleotides each containing the sugar ribose and one of the four adenine (A), guanine (G) uracil (U) or cytosine (C).
Oligo means a short sequence of DNA or DNA derivative typically 8 to 35 nucleotides in length. The exact size of the molecule will depend on many factors, which in turn depend on the ultimate function or use of the oligonucleotide. An oligonucleotide can be derived synthetically, by cloning or by amplification. The term “derivative” is intended to include any of the above described variants when comprising additional chemical moiety not normally a part of these molecules. These chemical moieties can have varying purposes including, improving a molecule's solubility, absorption, biological half life, decreasing toxicity and eliminating or decreasing undesirable side effects.
Autoimmune and inflammatory disease as used herein means diseases that are associated with autoimmune and inflammatory conditions such as inflammatory and autoimmune conditions such as osteoarthritis, reperfusion injury, asthma, multiple sclerosis, Guillain-Barre syndrome, Crohn's disease, ulcerative colitis, psoriasis, graft versus host disease, systemic lupus erythematosus, rheumatoid arthritis, Alzheimer's disease, toxic shock syndrome, insulin-dependent diabetes mellitis, acute and chronic pain as well as symptoms of inflammation and cardiovascular disease, stroke, myocardial infarction alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, Grave's disease, myasthenia gravis, scleroderma and atopic dermatitis.
Cardiovascular diseases as used herein means diseases that are associated with disorders of the cardiovascular system. Cardiovacular diseases can include but is not limited to the following disease conditions: heart failure, hypertension, coronary artery diseases, atherosclerosis, stroke/reperfusion and therapeutic angiogenesis.
Cell Line—refers to cells capable of stable growth in vitro for multiple generations.
Degranulation—refers to the movement and exocytosis of secretory lysosomes in cells having secretory lysomes.
Secretory Lysosomes—refers to structures found in mast cells, goblet cells, neutrophils, natural killer cells, basophils, eosinophiles, melanocytes, dendritic cells, macrophages, B cells, osteoclasts and platelets that are a mixture of lysosomes and secretory granules. By “secretory lysosome” it is meant a dual-function organelle that is used as both the lysosome (for degradation) and for storage of secretory proteins of the cell and which shares many features with both conventional lysosomes and secretory granules, such as structure and content. The terms secretory lysosomes and secretory granules are meant to be used interchangeably.
Activation—refers to the process in cells with secretory lysosomes whereby the cells upon presentation of an activating agent undergo a modification in gene expression and release of the contents of their secretory lysosomes. The term “activation” is intended to encompass the period from presentation or exposure to an activating agent until recovery
Activating agent—The term “activating agent” includes any chemical, physical, biological, electrical or radiation treatment, stimulus or condition which is capable of causing activation of cells having secretory lysosomes. In the case of mast cells an activating agent causes mast cell activation disease states and infection also may be considered an activating agent. Agents may also be inert or substances believed to be inert with the invention establishing the inertness such as proving pharmaceutically acceptable carriers are truly acceptable. It is understood that different activating agents will be used depending on the type of secretory lysosomal cell used and the selection and use of suitable activating agent are known to those skilled in the art. Specific activating agents provided in the instant invention include but are not limited to anti-IgE, DNP-HSA antibodies, adenosine, histamine, chemokines, stem cell factor, neuropeptides, complement factor proteins, C5a, natural or synthetic ligands for TLR receptors, compound 48/80.
Activation phase—the term “activation phase” refers to a particular stage of the activation of cells having lysosomal contents in response to an activating agent. An activation phase may correspond to a distinct cellular or metabolic event such as the onset of gene expression of a subset of genes or the secretion of a protein or other lysosome contents. The term activation phase as used herein is also understood to be descriptive of a temporal stage of activation (i.e. early, middle and late phases). In the case of mast cell activation the activation phases can be early, middle and late stages of mast cell activation. The early stage corresponds to between about 5 minutes and 2 hours after administration of an activating agent. The early stage is typically associated with degranuation. The middle stage corresponds to between about 2 and 16 hours after administration of an activating agent. The middle stage is generally associated with granule biosynthesis. The late stage corresponds to between about 16 and 48 hours after administration of an activating agent and generally associated with cytokine release which follows degranulation and secretory cell homeostasis.
Target—refers to any gene perturbed in a disease state, developmental stage or drug treatment. Frequently a target refers to a drug development target that is capable of being altered by an agent or compound. Such drug development targets are suitable for screening candidate compounds used in direct binding assays.
Hybridization—Association of two complementary nucleic acid strands or analogues thereof to form a double stranded molecule which can contain two DNA strands, two RNA strands, or one DNA strand and one RNA strand.
Up regulated—refers generally to an increase in level of gene expression normally in the response to an activation agent as herein defined. The expression of a gene is considered up regulated if the level of expression is at least 120 percent relative to control, preferably 150 percent relative to control and most preferably 200 percent or higher relative to control.
Down regulated—refers generally to a decrease in the level of gene expression normally in response to an activation agent as herein defined. The expression of a gene is considered down regulated if the level of expression is less than 80 percent relative to control, preferably less than 60 percent relative to control and most preferably 50 percent or lower relative to control.
DNA Microarray—refers collectively to a technique(s) used to measure and analyze the expression of a large number of genes simultaneously and as described in Microarray analysis Schena, Mark Wiley-Liss, 2003 incorporated heerin by reference. The term can refer to DNA microarrays which contain microscopic spots of about 1 kb DNA sequences representing thousands of genes bound to the surface of glass microscopic slides. The term can also refer to oligonucleotide arrays (DNA chips) or high density nucleotide probes which contain synthetic oligonucleotides representing thousands of gene sequences synthesized on the surface of small areas of a glass slide.

The method of the invention provides a general approach to study activation in cells having secretory lysosomes such as mast cells, goblet cells, neutrophils, natural killer cells, basophils, eosinophils, melanocytes, dendritic cells, macrophages, B cells, osteoclasts and platelets. The preferred method of the invention uses mast cells. The invention provides a method to identify and analyze genes that modified (i.e. up-regulated or down-regulated) at different activation stages in cells having secretory lysosomes. Using the method of the invention, many aspects of the activation of cells having secretory lysosomes can be studied in a single experiment/method. The method of the invention provides data on gene expression at one or more activation phases. Data obtained through the use of the method can provide a rationale to prioritize genes as candidates for target validation in the field of inflammation and autoimmune disease.

A large number of newly identified genes in the human genome show no significant sequence similarity to genes with known function. Therefore, these genes are not easily recognized as drug targets. Expression analysis is an alternative method to suggest a possible function for a given gene. (Mini Rev. Med. Chem.(2001) 1:197-205). The link between modulation of gene expression resulting in phenotypic or functional changes is well established. In mast cells for example, the mRNA levels for the early response gene c-fos is highly elevated within 1 h following mast cell activation (Cell Signal. 9: 65-70; 1997). This increase in mRNA levels is followed by increased protein levels and increased activity of this protein as measured by c-fos binding at AP-1 sites of DNA promoter regions. Another well known example in mast cells is the transcriptional regulation of the histidine decarboxylase (HDC) gene. HDC is the enzyme responsible for the biosynthesis of histamine. HDC is up-regulated in the mid to late phase of mast cell activation. The increase in HDC mRNA levels leads to an increase in HDC protein levels, which is associated with increased HDC activity and histamine levels. Immunology (2000) 99:600-604. Thus, novel methods aimed at studying temporal gene expression in mast cells as described herein are likely to lead to the identification of genes with functional relevance to mast cell physiology. Many of these functionally important genes will play a role in immunological and inflammatory responses.

The present invention also provides a method for finding novel genes and/or novel functions for known genes. In the case of mast cells of particular interest are genes having one or several of the following characteristics including but not limited to i) involvement in the IgE/antigen response or mast cell activation in general; ii) being involved in mast cell degranulation (early phase); iii) being involved in the late phase response; iv) being involved in mast cell re-granulation/granule biosynthesis; v) genes involved in mast cell homeostasis. The method of the invention can be used to identify genes having the properties listed above.

In the method of the invention, the level of gene expression in cells exposed to an activating agent is compared to the level of gene expression of control cells that have not been treated with the activating agent (control) at one or more activation phases. In the case of mast cells, the level of gene expression is measured at distinct activation phases. It is also contemplated that more than one measurement of expression levels can be determined in an individual activation phase. An activation phase is measured on the amount of time elapsed after administration of an activating agent to the experimental cells. Although samples can be collected at any time during or after mast cell activation samples are preferably collected at early phase, generally 5 minutes to 2 hours, middle phase at 6 to 12 hours, and late phase at 12 to 48 hours. The most preferred time points are at the early stage from 30 minutes to 2 hours, the middle stage from 6 to 12 hours and the late stage from 12 to 24 hours after administration of the activating agent. Data on the level of gene expression at varying time points after mast cell activation can provide information about the function of the genes as discussed herein.

The source of the cells can be tissues, tissue culture cells, cell extracts. Material collected from any of the sources is collectively referred to as “cells.” Preferably, the cells are obtained from tissue cell cultures and most preferably cells of mast cell lines such as MC/9 or HMC-1 (human mast cell line-1) cells. Cultured primary mast cells such as human lung derived mast cells are also preferred. The most preferred source of cells are rat basophilic RBL-2H3 cells. The cells are generally cultured for about 16 hours in OPTI-MEM medium before an activating agent is administered. In other embodiments of the invention the mast cells can be obtained from different sources such as primary or cultured human lung derived, human or mouse bone marrow derived, embryonic stem cell derived, cord blood derived, and different mast cell lines such as HMC-1 and MC/9.

The activating agent used can be a chemical, physical, biological, electrical or radiation treatment or a condition that is capable of producing a biological response such as mast cell activation. The preferred activating agents are chemical and biological agents and activation of mast cells can be obtained through stimulation of cell surface receptors such as an IgE molecule bound to its receptor, chemokine receptors, G protein coupled receptors (GPCRs), transporters and ion channels and toll like receptors. In the case of the most preferred embodiment the activation agent DNP-HSA (antigen for anti-DNP-HSA IgE) is administered to cells pre-incubated with the anti-DNP-HSA IgE antibody. Hong-Geller et al (2000) J. Cell Biol. 148:481-493. When the DNP-HSA activating agent is used, the experimental cells are exposed for between 2 and 120 minutes and most preferably between 5 and 30 minutes. Activating agents should be used in the manner and or amounts to promote the biological response. In the case of mast cells, activating agents are used in such a manner as to promote mast cell activation. In another embodiment of the invention, molecule antigen combinations could be used such as different IgE molecules/antigen combination and anti-IgE receptors for cross linking.

Levels of gene expression are determined from analysis of RNA isolated from cells and/or tissues after administration of an activating agent.

Methods of RNA isolation are well known in the art and the RNA isolation method used should depend on the source of the cells. See Maniatis et al, Molecular Cloning: A laboratory Manual, Third Edition (2001) (Cold Spring Harbor Press, Cold Spring Harbor, N.Y.). The preferred method of RNA isolation is the Qiagen RNA purification kit. (Qiagen, Valencia, Calif.).

Steps should be taken to avoid degradation of the RNA prior to analysis. Typically, RNA is isolated from cells soon after the cells have been collected for analysis. Cells that have been collected should be stored under conditions that limit the degradation of RNA known to those skilled in the art. Likewise, after RNA has been isolated from the cell samples the RNA should be stored under conditions that reduce RNA degradation. For example, RNA should be stored on dry ice or under −70° C. conditions under RNAse free conditions. DEPC water should be used in buffers and solutions. Conditions should also be maintained such that additional RNA synthesis is terminated when the cells are collected. In this way RNA expression will be representative of the types and levels of RNA expression at the time of collection.

Isolated RNA from the cells is used to synthesize double stranded DNA in a reverse transcriptase reaction that can be performed according to methods known to those skilled in the art. The preferred reverse transcriptase is the Superscript reverse transcriptase (Superscript Choice™, Invitrogen Carlsbad, Calif.). It is used according the manufacturers instructions. Approximately 5 to 15 μg total RNA from each time points are used to measure in reverse transcriptase reactions. The amount of RNA used varies depending on the number of genes tested and the method used to detect gene expression.

The cDNA is used as a template for the synthesis of labeled cRNA with a plasmid or vector. The cRNA can be labeled with fluorescence or with other methods commonly used in the art such as for labeling nucleic acids. The cRNA is most preferably labeled with biotin. The cRNA is then fragmented using an alkaline base method commonly used in the art.

Analysis of Gene Transcription

RNA levels can be measured using a number of techniques available to those skilled in the art. Quantitative methods for detecting specific RNA levels of certain genes can be used such as Northern hybridization, PCR analysis, or microarray analysis. The preferred method of RNA analysis is microarray analysis.

Laboratory materials and equipment for performing microarray analysis are available from companies such as Affymetrix, Agilent and Spotfire. Microarray or cRNA chip analysis offer the advantage of being able to analyze multiple genes in a single experiment. Preparation of cRNA, hybridization are performed according to methods commonly used in the art. Microarray analysis can be performed using procedures available from various companies such as Affymetrix and Spotfire.

The Affymetrix procedure is the preferred method. The samples can be hybridized to the human genome U133 microarray which is comprised of two microarrays over 1,000,000 oligonucleotides covering more than 39,000 transcript variants representing 33,000 human genes. The samples can also be hybridized to the Rat genome U34 set which contains more than 24,000 known genes and EST clusters. The U34 array consists of U34A, U34B and U34C chips and can be performed essentially as follows: Between 5 and 15 μg of the total RNA can be converted into double stranded cDNA by reverse transcription using a cDNA synthesis kit. The preferred kit for cDNA synthesis is Superscript Choice™, Invitrogen (Carlsbad, Calif.) which has a special oligo (dT024 primer) (Genset, La Jolla, Calif.) containing a T7 RNA polymerase promoter site added 3′ of the poly T tract. After second strand synthesis, labeled cRNA is generated from the cDNA samples by an in vitro transcription reaction using a reporting reagent such as biotin-11-CTP and biotin-16-UTP (Enzo, Farmingdale, N.Y.). Labeled cRNA can be purified by techniques commonly used in the art. The preferred method is to use RNeasy spin columns (Qiagen, Valencia, Calif.). Current Protocols in Molecular Biology, John Wiley and Sons, July, 2002. About 5 to 30 micrograms of each cRNA sample can be fragmented by mild alkaline treatment. Preferably, the cRNA sample is fragmented by treatment at 94° C. for 35 minutes in fragmentation buffer as suggested by the manufacturer. A mixture of control cRNAs for bacterial and phage genes was included to serve as tools for comparing hybridization efficiency between arrays and for relative quantitation of measured transcript levels. Before hybridization, the cRNA samples can be heated at about 94° C. for 5 minutes, equilibrated at 45° C. for 5 minutes and clarified by centrifugation (14,000×g) at room temperature for 5 min. Aliquots of each cRNA sample are hybridized to arrays, or stored according the manufacturer's directions. The arrays are then washed according to methods commonly used in the art. The preferred wash is with non-stringent (6×SSPE, 0.01% Tween-20, 0.005% antifoam) and stringent (100 mm MES, 0.1M NaCl, 0.01% Tween 20), stained with R- Phycoerythrin Streptavidin- (Molecular Probes, Eugene, Oreg.), washed again and scanned by an argon-ion laser scanner with the 560-nm long-pass filter (Molecular Dynamics; Affymetrix). Data analysis can be performed in order to determine if a gene expression level is increased or decreased or unchanged. Preferably, software such as MAS4.0 or MAS 5.0 software (Affymetrix, Calif.) is used for data analysis.

A gene would be considered to have modified expression in activated cells with secretory lysosomes if the expression profile of the gene indicates that it is either up regulated or down regulated as the terms are defined herein. It is understood that when measuring expression levels using microarray analysis that the level of expression is reproducibly above the noise levels obtained from measurement of gene expression with a microarray apparatus. The noise level can vary depending on variables (such as quality of cRNA probes, sensitivity of detection and quality of oligos on the chip) that effect noise level

For instance, a gene would be considered to have modified expression in mast cell degranulation (early phase) if the gene were up regulated during the early activation phase, typically measured at 2 h. A gene would be a preferred candidate for involvement in mast cell degranulation if the expression levels returned to normal levels relative to control during the late phase of activation.

It is also contemplated that the method of the invention can be used with other activation agents such as neuropeptides, chemokines, cytokines, small molecule activators of mast cell function such as compound 40/80, agonists or antagonists for receptors expressed at the surface of mast cells.

Preferred Embodiment of the Invention

The following examples are provided to illustrate the invention, but not to limit its scope. Other variants of the invention will be readily apparent to one of ordinary skill in the art. The contents of all references, patents and published patent applications cited throughout this application, as well as the figures and sequence listing are hereby incorporated by reference.

The activation agents, IgE and DNP-HSA were administered to RBL-2H3 cells and cells were collected at time points of 0 hour (h) (control), 2 h, 6 h and 24 h. The 2 h time point is expected to yield primarily early response genes and genes linked to the early phase of the allergic response. The 6 h time point should uncover genes involved in granule biosynthesis in the middle and in the late phase allergic response. The 24 hour time provides information on genes that are involved in granule biosynthesis and mast cell homeostasis.

Prior to antigen stimulation, the RBL 2H3 cells were incubated for 2 h at 37° C. with anti-DNP IgE (Spe-1 clone ATCC# CTL-2256) at a final concentration of 2 μg/ml in OPTI-MEM/10% FBS. Cells were washed twice with Tyrode's Buffer (at 37 C) and then DNP-HSA (DNP coupled to human serum albumin; 100 ng/ml in Tyrode buffer) was added at a final concentration of 100 ng/ml. The antigen stimulation times were 0, 2, 6 and 24 h. At the end of each time point an aliquot of (1 ml) of the culture media was kept for the determination of hexosaminidase enzymatic activity. The remaining media was discarded and cells were washed once with 2 ml ice cold PBS and were then processed for RNA extraction as described below.

Hexosaminidase Assay

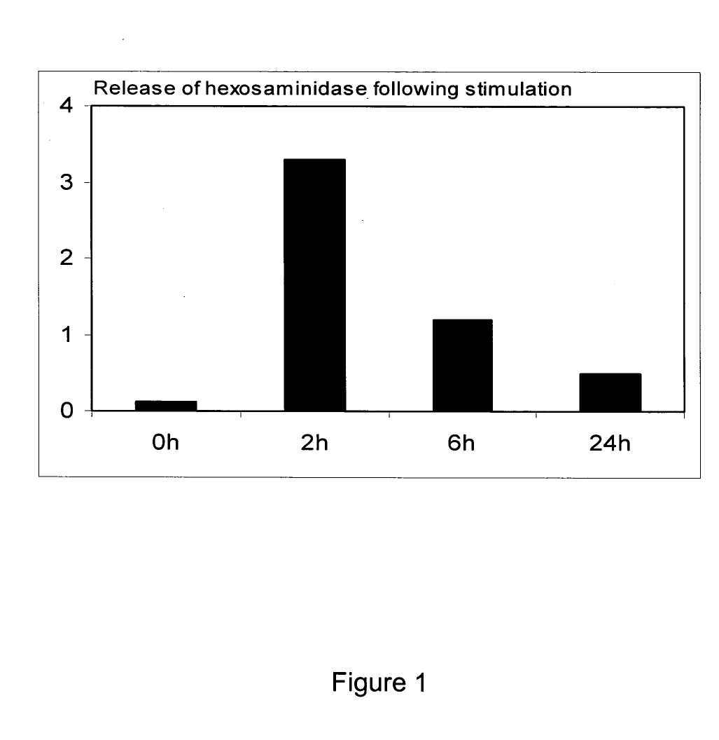

The hexosaminidase assay was performed to determine the level of mast cell activation. This protocol was adapted from Schwartz et al. Journal of Immunology 123: 1445-1450; 1979. The reaction mixtures were prepared in a 96 well plate using 10 μl of culture supernatant and 50 ul of 4 mM p-nitrophenol-β-D-2-acetamido-2-deoxyglucopyranoside (Sigma) in 0.04 M citrate buffer (buffer titrated to pH 4.5 with 0.2M dibasic sodium phosphate). As a negative control, 10 μl of Tyrode's buffer was used. After mixing, the plate was incubated at 37° C. in a humidified incubator for 90 min. The reaction was stopped by adding 150 μl of 0.2 M glycine pH 10.7. The samples were read on a SPECTRAmax 340 plate reader (Molecular Devices, Sunnyvale, Calif.) at a wavelength of 410 nm. The release of hexosaminidase following stimulation is shown in FIG. 1.

Preparation of cRNA

RNA was isolated from the samples using the RNAeasy total RNA isolation kit from Qiagen as described by the manufacturer. The homogenization solution was added directly to the cell monolayer and homogenates were processed as recommended by the manufacturer.

Between 5-10 μg of the total RNA was converted into double stranded cDNA by reverse transcription using a cDNA synthesis kit (Superscript Choice, Invitrogen) with a special oligo (dT024 primer) (Genset, La Jolla, Calif.) containing a T7 RNA polymerase promoter site added 3′ of the poly T tract. SEQ. ID. No. 1.

Preparation of cRNA was performed according to the manufacturer's protocol (Affymetrix, Santa Clara, Calif.). After second strand synthesis, labeled cRNA was generated from the cDNA samples by an in vitro transcription reaction supplemented with biotin-11-CTP and biotin-16-UTP (Enzo, Farmingdale, N.Y.). The labeled cRNA was purified by using RNeasy spin columns (Qiagen, Valencia, Calif.). Fifteen micrograms of each cRNA sample was fragmented by mild alkaline treatment at 94° C. for 35 minutes in fragmentation buffer (40 mM Tris-acetate, pH 8.1,100 mM potassium acetate, 30 mM magnesium acetate) and then used to prepare 0.3 ml of master hybridization mix (100 mM MES, 1M [NaCl], 20 mm EDTA, 0.01% Tween 20, 0.1 mg/ml herring sperm DNA (Promega, Madison, Wis.), 0.5 mg/ml acetylated BSA (Invitrogen)). A mixture of control cRNAs, available from the manufacturer, for bacterial and phage genes was included in the mix (BioB, BioC, BioD, and cre, at 1.5, 5, 25 and 100 pM, respectively) to serve as tools for comparing hybridization efficiency between arrays and for relative quantitation of measured transcript levels. Before hybridization, the cRNA samples were heated at 94° C. for 5 minutes, equilibrated at 45° C. for 5 minutes and clarified by centrifugation (14,000×g) at room temperature for 5 min.

Hybridization Analysis and Scanning of the Rat U34 Arrays

RNA isolated from the samples was hybridized to the Rat U34 microarray. The Rat genome U34 set contains more than 24,000 known genes and EST clusters. The U34 array consists of U34A, U34B and U34C chips. The hybridizations were performed in the following manner and follow the manufacturer's protocol (Affymetrix, Santa Clara, Calif.). Aliquots of each sample (10 μg of cRNA in 200 μl of the master mix) were hybridized to Rat U34 arrays (Affymetrix) at 45° C. for 16 h in a rotisserie oven set at 60 rpm. The arrays were then washed with non-stringent (6×SSPE, 0.01% Tween-20, 0.005% antifoam) and stringent (100 mm MES, 0.1M NaCl, 0.01% Tween 20), stained with R-Phycoerythrin Streptavidin- (Molecular Probes, Eugene, Oreg.), washed again and scanned by an argon-ion laser scanner with the 560-nm long-pass filter (Molecular Dynamics; Affymetrix).

Data Analysis

Data analysis was performed by using GENECHIP 3.2 software. The software includes algorithms that determine whether a gene is absent or present (absolute call) and whether the expression level of a gene in an experimental sample is significantly increased or decreased (difference call) relative to a control sample. To assess differences in gene expression, genes were selected genes based on fold change at 2 fold or more in conjunction with absolute call and difference call. Specifically, the following criteria were selected for significant changes for primary screen of each time point: (1) the change in the average difference across all probe sets was >2 fold; (2) for induced genes, a difference call of “increase” or “marginal increase” should be present, and an absolute call of “presence” should be associated with the experimental sample; (3) for suppressed genes, a difference call of “decrease” or “marginal decrease” should be present, and an absolute call of “presence” should be associated with the control sample.

Results and Discussion

Using the method of the invention, it was found that the expression of many genes was increased or decreased after IgE stimulation as shown in Table 1, suggesting a role for these genes in mast cell function.

TABLE I
Numbers of gene hits (2-fold or more)

| Time after addition | | |
|---|---|---|
| activation agent | Up-regulated | Down-regulated |
| 2 h | 155 | 177 |
| 6 h | 140 | 566 |
| 24 h | 306 | 374 |

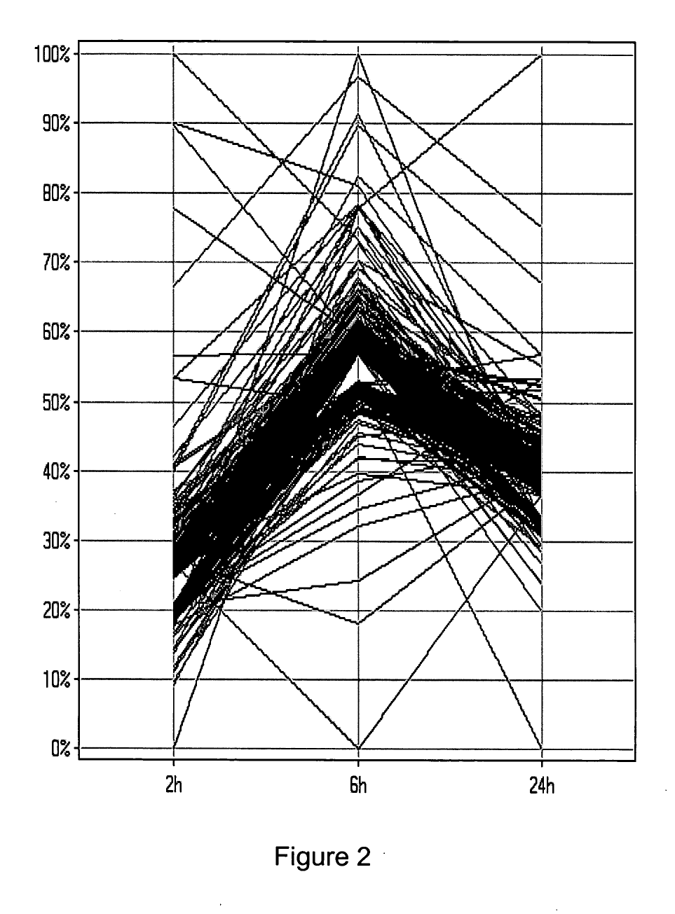

In order to identify genes essential for mast cell physiology the temporal peak for transcriptional activation and repression following mast cell activation are determined. Our method allows for the temporal analysis of mast cell gene expression profiles following activation. FIG. 2 shows the expression profiles at 2 h, 6 h, and 24 h after mast cells have been stimulated with IgE/antigen. Each line represents the relative expression profile (in percentage) of a gene with 2-fold or more induction compared to the unstimulated control (see methods). The data shows that most up-regulated genes will show upregulation at 2 h, peak after 6 h, followed by a decreased in mRNA level at 24 h. This important information was not available in the current mast cell literature.

Gene Expression Peaking at 2 h

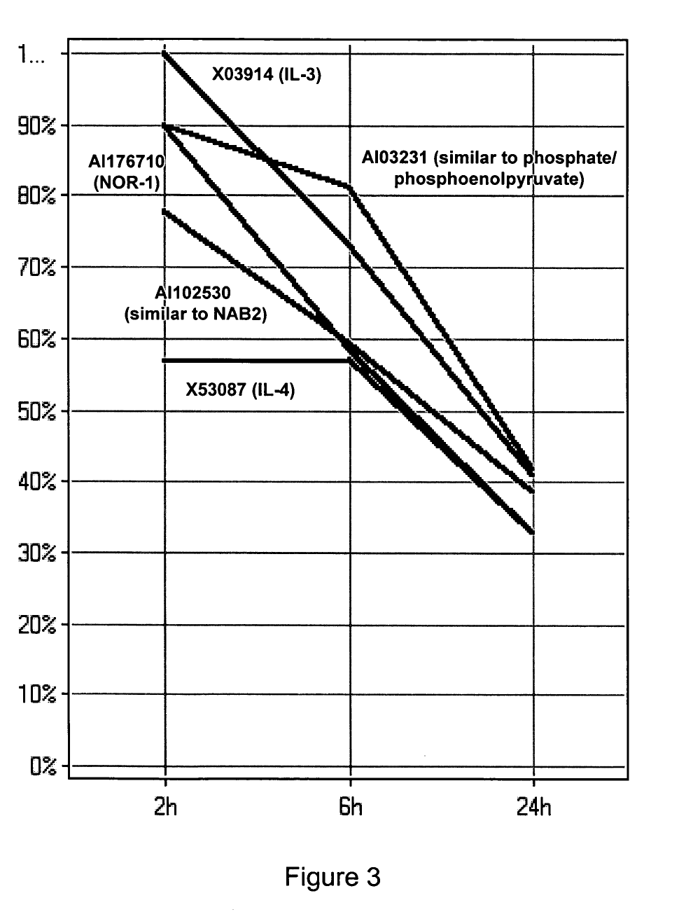

Genes showing peak up regulation relative to T(o) at 2 h or less are considered early response genes. Once translated, these genes will contribute directly to the late phase. These genes can also contribute indirectly (in trans) to the transcriptional activation of late phase genes. FIG. 3 shows the profile of genes (from FIG. 2) with peak expression at 2 h. The gene profiling was done using the Spotfire software. Two of these genes are the cytokines IL-3 (accession # X03914) and IL-4 (accession # X53087). The two cytokines are known to be up regulated rapidly after IgE/DNP-HSA stimulation of mast cells. Toxicology (1997) 116: 211-218.

Another gene with peak expression at 2 h is Nor-1 (accession # AI176710). Nor-1 had expression of 38-fold or 3800% relative to t(o). Nor-1 is a member of the orphan nuclear receptor family and is also known to be an early response gene (JBC 277:33001-33011; 2002). Interestingly, its expression has never been reported in mast cells. The expression profile of Nor-1 suggests that it may be a master gene in the control of transcriptional regulation in mast cells. Modulating Nor-1 activity with natural or synthetic ligands could lead to therapeutic intervention in the field of allergy, immunological diseases or cardiovascular diseases.

Several ESTs show a peak induction at 2 h after stimulation. For example, an EST with high similarity to a phosphate/phosphoenolpyruvate translocator (accession # AI103231) was identified. The presence of this EST (or related genes) and its transcriptional regulation in mast cells was not documented previously. Phosphate translocators are known to couple to ATP pumps in chloroplast of plants. Proton pumps are responsible for generating electrical and chemical gradients across organelle membranes with the magnitude of these gradients ultimately determined by both ions and organic solute transporter located in the vesicle membrane. Phosphate translocators are members of these solute transporter. Thus, using the method of the invention, an EST encoding a gene with a potentially important role in the biosynthesis and regulation of mast cell granules was identified. Regulating the activity of the transporter by small molecules could impact granule or secretory lysosomes biosynthesis and in turn modulate mast cell physiology.

Gene Expression Peaking at 6 h

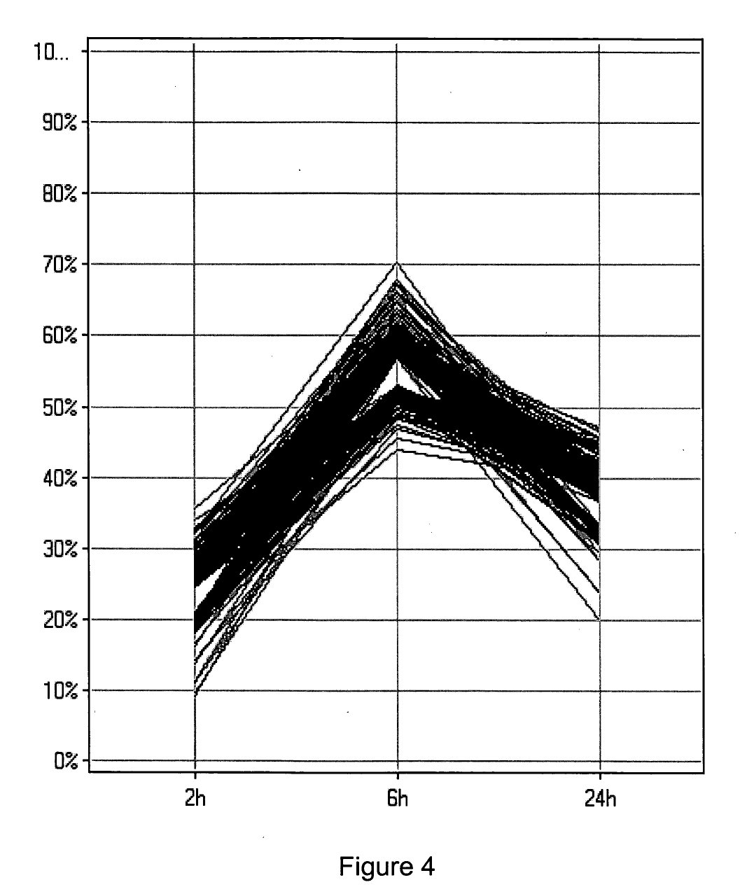

A large proportion of the genes up or down regulated following mast cell activation peaked 6 h after stimulation (FIG. 4). This suggests that the stage following the early phase of activation but preceding the steady state (more than 24 h) is crucial for transcriptional regulation of mast cell effector genes. Our data suggests that many genes known to be important in mast cell physiology are up regulated at the 6 h time point. Several examples are listed below.

Several papers show that sphingosine kinase is pivotal to the activation of signaling cascades initiated at the Fcε receptor. It was proposed that the balance between sphingosine and sphingosine-1-phosphate is decisive for mast cell activation after Fcε receptor triggering. Prieschl, E. E. et al (1999) J. Ex. Med. 190:1-8. Sphingosine kinase catalyzes the conversion of sphingosine to sphingosine-1-phosphate. Sphingosine-1-phosphate (SIP) is formed in response to diverse stimuli (cytokines, growth factors, antigens, G-coupled receptors). Once produced, SIP can act as a second messenger. SIP can also be secreted and act in an autocrine/paracrine manner. Our data shows that the sphingosine kinase gene (AI105383) is upregulated 3-fold following mast cell activation with its peak induction at 6 h. Selectively blocking sphingosine kinase gene expression or inhibiting its kinase activity are potential therapeutic approaches in mast cell associated diseases.

By using the method of the invention it has also been shown that relaxin is up-regulated 3-fold, 6 h after IgE/DNP stimulation of RBL. Relaxin was also found to be up-regulated after mast cell activation by another group. Chen, H. et al (1998) J. Exp. Med. 188:1657-68. Relaxin is a 6 kDa polypeptide hormone (insulin family) predominantly produced by the corpus luteum during pregnancy. More recently, it was shown that relaxin can modulate the activity of bone marrow-derived cells, such as mast cells, platelets and granulocytes. In mast cells, relaxin can inhibit degranulation (histamine release). Relaxin can also reduce the allergic asthma-like reactions elicited by antigen inhalation in sensitized guinea pigs. J. Clin. Invest. (1994) 94:1974-80. More recently, relaxin was shown to favor the development of activated human T cells into Th1-like effectors. Relaxin or relaxin receptor agonist could be useful therapeutic modalities.

Using the method of the invention many ESTs with peak expression at 6 h have been identified. The corresponding genes are likely to play a role in regulating mast cell activation. Moreover, we found known genes up-regulated or down regulated at 6 h with no known function in mast cell physiology. A few examples are listed below.

The gene encoding epithelial membrane protein-1 (EMP- 1; Z54212) is upregulated 20-fold at 6 h. The peripheral myelin protein 22 (PMP22) and the epithelial membrane proteins (EMP- 1, -2, and -3) comprise a subfamily of small hydrophobic membrane proteins. The putative four-transmembrane domain structure as well as the genomic structure are highly conserved among family members. PMP22 and EMPs are expressed in many tissues, and functions in cell growth, differentiation, and apoptosis have been reported. EMP-1 is highly upregulated during squamous differentiation and in certain tumors, and a role in tumorigenesis has been proposed. The P2X7 protein, which is an integral cation-permeable channel, was recently shown to interact with the EMPs (J Biol Chem. (2002) 277:34017-23. The fact that EMP-1 is upregulated 20-fold after IgE stimulation and its ability to regulate channels suggest it might play a role in mast cell physiology.

Our data shows 4-fold up-regulation of a gene weakly similar to the G-coupled receptor VTR-15-20 (AI172577). VTR 15-20 mRNA is expressed in brain and spleen and is regulated by immunologic challenge. Charlton M E., (1997) Brain Res 764:141-148. Based on the cellular distribution and regulation by immune challenge, VTR 15-20 was hypothesized to play a role in neuro-immune function. Given that mast cells are also known to play a role in the neuro-immune junction, the VTR15-20 homolog identified by our method could be crucial for that function.

Gene Expression Peaking at 24 h

An important aspect of mast cell physiology is that after degranulation, mast cells will re-synthesize their granule content in preparation for the next round of degranulation. Thus, genes that are slowly (24 hours or more) up or down regulated after degranulation are most likely part of mast cell homeostasis and critical to maintain mast cell phenotype. Current therapies in allergies target the acute/early phase of the response but many potential targets could be found by focusing on the re-granulation process. For example, interfering with re-granulation would prevent or reduce the chronic component of allergic diseases. The method of the invention allows us to follow gene expression at different time points and to identify genes involved in mast cell homeostasis.

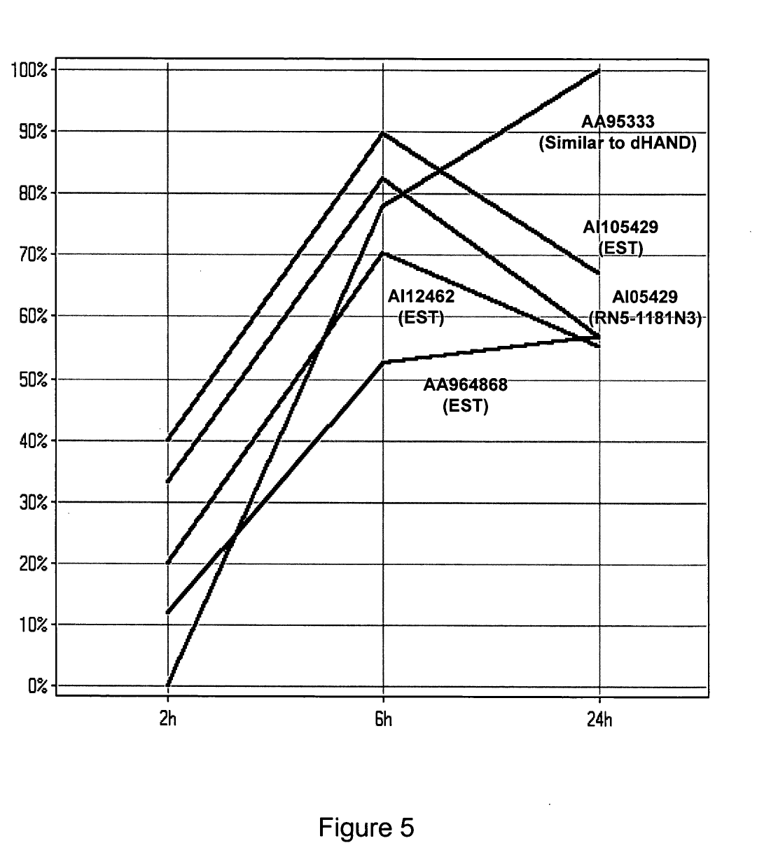

As shown in FIG. 5, several genes peaked at 24 h following IgE/antigen stimulation. Interestingly, all these genes are ESTs. Our data confirms that this phase of mast cell activation as been largely overlooked and outlines the importance of studying kinetic expression profiles rather than unique time points. Out of the 8 gene profiles in FIG. 4, only one (AA957333) has homology to a known gene. This EST has weak homology to the dHand gene. dHand is a transcription factor of the basic helix-loop-helix (bHLH) family; it is involved in cardiac and limb development. Mcfadden, DG (2002) Development 129:3077-3088. The gene corresponding to the EST AA957333 could encode for a protein involved in mast cell differentiation and maintenance of the mast cell phenotype.

| | |
|---|---|
| T7-(dT)24 Primer Sequence: | |
| 5′-GGCCAGTGAATTGTAATACGACTCACTATAGGGAGGCGG-(dT)24-3′ | SEQ. ID. No. 1 |

1. A method of identifying and analyzing genes having modified expression in activated cells with secretory lysosomes comprising the steps of:
a) exposing experimental cells to an activating agent;
b) preparing RNA from said experimental cells at one or more activation phases;
c) measuring the level of gene expression in the cells;
d) comparing the levels of gene expression of said experimental cells to the level of gene expression in control cells that have not been exposed to an activating agent;
e) identifying genes that are up regulated or down regulated in said experimental cells relative to said control cells.

2. The method of claim 1 using cells having secretory lysosomes selected from the list consisting of mast cells, goblet cells, neutrophils, natural killer cells, basophils, eosinophils, melanocytes, dendritic cells, macrophages, B cells, osteoclasts or platelets.

3. The method of claim 2 wherein the wherein the cells with secretory lysosomes are mast cells.

4. The method of claim 3 wherein mast cells are selected from the list consisting of MC/9, HMC-1, primary mast cells, cord blood derived mast cells, RBL 2H3 cells, bone marrow derived mast cells, ES cells derived mast cells, or lung-derived mast cells.

5. The method of claim 4 wherein the cells are mast cells obtained from a rat basophilic RBL 2H3 cell line.

6. The method of claim i wherein the activation phases are selected from early, middle and late stages of activation in cells with secretory lysosomes.

7. The method of claim 6 wherein the early phase is from about 5 minutes and 2 hours after administration of an activation agent.

8. The method of claim 7 wherein the early phase is from about 30 minutes to 2 hours after administration of an activation agent.

9. The method of claim 6 wherein the middle stage is from 2 and 16 hours after administration of the activation agent.

10. The method of claim 9 wherein the middle stage is from 6 to 12 hours after the administration of the activating agent.

11. The method of claim 6 wherein the late stage is from 16 and 48 hours after administration of the activating agent.

12. The method of claim 11 wherein the late stage is from 12 to 24 hours after administration of the activating agent.

13. The method of claim 1 wherein said activating agent is comprised of an antigen capable of causing activation of cells with secretory lysosomes.

14. The method of claim 1 wherein said activating agent is selected from the list consisting of activating anti-IgE antibodies, DNP-HSA, adenosine, histamine, chemokines, stem cell factor, neuropeptides, complement factor proteins, C5a, natural or synthetic ligands for TLR receptors, compound 48/80.

15. The method of claim 14 wherein said activating agent is DNP-HAS in combination with IgE.

16. The method of claim 1 wherein the gene is up regulated if the level of gene expression is elevated at least 120 percent relative to control.

17. The method of claim 16 wherein the gene is up regulated if the level of gene expression is elevated at least 200 percent relative to control.

18. The method of claim 1 wherein the gene is down regulated if the level of gene expression is less than 80 percent relative to control.

19. The method of claim 1 wherein the gene is down regulated if the level of gene expression is less than 50 percent relative to control.

20. The method of claim 1 wherein levels of a gene expression are measured using microarray analysis.

21. A method of treating chronic inflammation in humans said method comprised of the step of administering to a human in need thereof a pharmaceuticals acceptable amount of an inhibitor of a gene that is identified using the method of claim 1.

22. A method of treating cardiovascular disease in humans said method comprised of the step of administering to a human in need thereof an inhibitor of a gene that is identified using the method of claim 1.

23. The method of claims 22 wherein the disease is selected from the list consisting of:
heart failure, hypertension, coronary artery diseases, atherosclerosis, stroke/reperfusion and therapeutic angiogenesis.
